(19) United States
(12) Patent Application Publication
(10) Pub. No.: US 2025/0222232 A1
(43) Pub. Date: Jul. 10, 2025

(54) ARTIFICIAL INTELLIGENCE-BASED CONTROL OF CATHETER MOVEMENT

(71) Applicant: Expanse Technology Partners, LLC, Pleasanton, CA (US)

(72) Inventors: Eitan Konstantino, Orinda, CA (US); Tanhum Feld, Moshav Merhavya (IL); Stephanie Morgan Boula, Fremont, CA (US); Shawn McGuire Carter, Santa Rosa, CA (US); Ernest William Heflin, Pleasanton, CA (US); Jonah Shifrin, Chicago, IL (US)

(21) Appl. No.: 18/853,035

(22) PCT Filed: Apr. 3, 2023
(86) PCT No.: PCT/US2023/017314

§ 371 Date: Sep. 30, 2024

Related U.S. Application Data
(60) Provisional application No. 63/328,192, filed on Apr. 6, 2022.
(60) Provisional application No. 63/448,949, filed on Feb. 28, 2023.

(51) Int. Cl. A61M 25/01; A61B 17/00; A61B 17/22; A61M 25/06; G05B 13/02

(52) U.S. Cl. CPC A61M 25/0113; A61B 17/22; A61M 25/0662; G05B 13/0265; A61B 2017/00039; A61B 2017/22079; A61B 2217/007; A61M 2025/0681; A61M 2205/3327

(57) ABSTRACT

An improved mechanical thrombectomy system is described herein that uses artificial intelligence to acquire, extrude, segment, and/or aspirate more effectively blood clots regardless of the age, size (e.g., length, diameter, etc.), solidness, or location of the blood clots while lowering the bleeding risk during the operation. For example, the improved mechanical thrombectomy system described herein can include a catheter, one or more sensors coupled to the catheter, and a control unit coupled to the catheter and sensor(s) that uses artificial intelligence to acquire, extrude, segment, and/or aspirate clots.

### RELATED APPLICATIONS

This application claims the benefit of U.S. Provisional Patent Application No. 63/328,192, entitled “ARTIFICIAL INTELLIGENCE-BASED CONTROL OF CATHETER MOVEMENT” and filed on Apr. 6, 2022, and U.S. Provisional Patent Application No. 63/448,949, entitled “ARTIFICIAL INTELLIGENCE-BASED CONTROL OF CATHETER MOVEMENT” and filed on Feb. 28, 2023, which are both hereby incorporated by reference herein in their entireties. This application is also related to U.S. patent application Ser. No. 17/658,244, entitled “ASPIRATION CATHETER” and filed on Apr. 6, 2022, which is hereby incorporated by reference herein in its entirety. Any and all applications for which a foreign or domestic priority claim is identified in the Application Data Sheet as filed with the present application are hereby incorporated by reference under 37 C.F.R. § 1.57.

### TECHNICAL FIELD

The present disclosure relates to the field of medical methods and devices, more specifically to a catheter operated by a motor that is controlled, in part, using artificial intelligence.

### BACKGROUND

Thromboembolism is a disease caused by blood clot formation. In the venous system, thromboembolism has two distinct peripheral manifestations—deep vein thrombosis (DVT) and pulmonary embolism (PE). Venous thromboembolism is a leading cause of death and disability worldwide and represents the third most common vascular diagnosis in the United States, after myocardial infarction and stroke. Researchers estimate that there are approximately one million venous thromboembolism patients in the United States annually, leading to 600,000 hospitalizations. This results in approximately 60,000-180,000 deaths in the United States, within the first 30 days, each year and an estimated venous thromboembolism-related direct health care costs exceed $10 billion per year.

Clots and their impact are, by their nature, heterogenous and unpredictable. Thrombi can have a variety of morphologies. Due the characteristics of the vascular system and clot morphology, by the time thromboembolism is diagnosed, the underlying clot can be significant in size and hardness due to age. As a result, methods designed to remove fresh, soft clots are inadequate and ineffective for removing the larger, older clots often associated with venous thromboembolism. Current products are cumbersome and deliverability is, in many cases, compromised due to rigid catheters and complex mechanical components.

### SUMMARY

The systems, methods, and devices described herein each have several aspects, no single one of which is solely responsible for its desirable attributes. Without limiting the scope of this disclosure, several non-limiting features will now be discussed briefly.

One aspect of the disclosure provides a system comprising a catheter comprising an outer sheath and an inner sheath. The system further comprises one or more sensors coupled to the catheter. The system further comprises a control unit coupled to the catheter and the one or more sensors. The control unit comprises a motor configured to move the inner sheath with respect to the outer sheath. The control unit further comprises a processor configured with computer-executable instructions, where the computer-executable instructions, when executed by the processor, cause the processor to: obtain sensor data from at least one of the one or more sensors; determine an amount of power consumed by the motor while moving the inner sheath with respect to the outer sheath; apply the sensor data and an indication of the amount of power consumed by the motor as an input to a trained artificial intelligence model, where application of the sensor data and the indication of the amount of power consumed by the motor as an input to the trained artificial intelligence model causes the trained artificial intelligence model to output an amplitude and a frequency; and cause the motor to adjust operation so that the inner sheath oscillates between a retracted position and a protracted position by a distance corresponding to the amplitude at the frequency.

The system of the preceding paragraph can include any sub-combination of the following features: where the computer-executable instructions, when executed by the processor, further cause the processor to apply the sensor data, the indication of the amount of power consumed by the motor, and a type of the motor as an input to the trained artificial intelligence model; where the catheter further comprises a valve at a distal end of the catheter configured to be inserted into a venous system; where the computer-executable instructions, when executed by the processor, further cause the processor to apply the sensor data, the indication of the amount of power consumed by the motor, and a type of the valve included in the catheter as an input to the trained artificial intelligence model; where the valve is closed when the inner sheath is in the protracted position; where the valve is open when the inner sheath is in the protracted position; where the trained artificial intelligence model is associated with at least one of a type of the valve or a type of the motor; where the one or more sensors comprises at least one of a flow sensor, a contact sensor, a temperature sensor, a pressure sensor, or a camera; where at least some of the one or more sensors are coupled to a distal end of the catheter configured to be inserted into a venous system; where at least some of the one or more sensors are coupled to a proximal end of the catheter toward which one or more blood clot pieces are configured to be aspirated during operation of the catheter; and where the system further comprises a catheter actuation training system configured with second computer-executable instructions, where the second computer-executable instructions, when executed, cause the catheter actuation training system to: train an artificial intelligence model using training data to form the trained artificial intelligence model, and cause the trained artificial intelligence model to be loaded onto a storage medium of the control unit.

Another aspect of the disclosure provides a computer-implemented method for actuating an inner sheath of a catheter. The computer-implemented method comprises: obtaining sensor data from at least one sensor coupled to the catheter; determining an amount of power consumed by a motor configured to move the inner sheath with respect to an outer sheath of the catheter while moving the inner sheath with respect to the outer sheath; applying the sensor data and an indication of the amount of power consumed by the motor as an input to a trained artificial intelligence model, where application of the sensor data and the indication of the amount of power consumed by the motor as an input to the trained artificial intelligence model causes the trained artificial intelligence model to output an amplitude and a frequency; and causing the motor to adjust operation so that the inner sheath oscillates between a retracted position and a protracted position by a distance corresponding to the amplitude at the frequency.

The computer-implemented method of the preceding paragraph can include any sub-combination of the following features: where applying the sensor data and an indication of the amount of power consumed by the motor as an input to a trained artificial intelligence model further comprises applying the sensor data, the indication of the amount of power consumed by the motor, and a type of the motor as an input to the trained artificial intelligence model; where the catheter further comprises a valve at a distal end of the catheter configured to be inserted into a venous system; where applying the sensor data and an indication of the amount of power consumed by the motor as an input to a trained artificial intelligence model further comprises applying the sensor data, the indication of the amount of power consumed by the motor, and a type of the valve included in the catheter as an input to the trained artificial intelligence model; where the valve is closed when the inner sheath is in the protracted position, and where the valve is open when the inner sheath is in the protracted position; and where the trained artificial intelligence model is associated with at least one of a type of the valve or a type of the motor.

Another aspect of the disclosure provides a non-transitory, computer-readable medium comprising computer-executable instructions for actuating an inner sheath of a catheter, where the computer-executable instructions, when executed by a computer system, cause the computer system to: obtain sensor data from at least one sensor coupled to the catheter; determine an amount of power consumed by a motor configured to move the inner sheath with respect to an outer sheath of the catheter while moving the inner sheath with respect to the outer sheath; apply the sensor data and an indication of the amount of power consumed by the motor as an input to a trained artificial intelligence model, where application of the sensor data and the indication of the amount of power consumed by the motor as an input to the trained artificial intelligence model causes the trained artificial intelligence model to output an amplitude and a frequency; and cause the motor to adjust operation so that the inner sheath oscillates between a retracted position and a protracted position by a distance corresponding to the amplitude at the frequency.

The non-transitory, computer-readable medium of the preceding paragraph can include any sub-combination of the following features: where the computer-executable instructions, when executed, further cause the computer system to apply the sensor data, the indication of the amount of power consumed by the motor, and a type of the motor as an input to the trained artificial intelligence model; and where the catheter further comprises a valve at a distal end of the catheter configured to be inserted into a venous system, and where the computer-executable instructions, when executed, further cause the computer system to apply the sensor data, the indication of the amount of power consumed by the motor, and a type of the valve included in the catheter as an input to the trained artificial intelligence model.

### BRIEF DESCRIPTION OF DRAWINGS

Throughout the drawings, reference numbers may be re-used to indicate correspondence between referenced elements. The drawings are provided to illustrate example embodiments described herein and are not intended to limit the scope of the disclosure.

FIG. 1 is a block diagram of an illustrative operating environment of a mechanical thrombectomy system in which a catheter control unit uses artificial intelligence to cause actuation of a catheter.

FIG. 2 is a flow diagram illustrating the operations performed by the components of the operating environment of FIG. 1 to determine an amplitude and frequency by which the inner sheath of the catheter is to be actuated.

FIGS. 3A-3B illustrate an example catheter in various positions.

FIGS. 4A-4D illustrate an example catheter being used in a procedure to aspirate a clot.

FIG. 5 is a flow diagram depicting an example, inner sheath actuation routine illustratively implemented by a catheter control unit, according to one embodiment.

### DETAILED DESCRIPTION OF SPECIFIC EMBODIMENTS

As explained above, methods designed to remove fresh, soft clots are inadequate and ineffective for removing larger, older clots often associated with venous thromboembolism. Current products are cumbersome and deliverability is, in many cases, compromised due to rigid catheters and complex mechanical components. In addition, while blood thinners can mitigate the risk of future clots, the use of blood thinners to remove clots typically takes a 12-24 hour-long procedure. Even then, blood thinners are often unable to break down or eliminate existing blood clots and can cause a significant increase in the bleeding risk to a patient.

Many solutions have been developed to treat thromboembolism-from open surgical methods to minimally-invasive catheter-based solutions-but such solutions are often limited when used in an attempt to treat thromboembolism. For example, a mechanical thrombectomy system is one type of solution that has been developed. However, typical mechanical thrombectomy systems are not equipped to deal properly with thromboembolism due to poor deliverability, typical clot geometries, typical clot volumes, and typical clot compositions. In particular, typical mechanical thrombectomy systems are generally too large in size, leading to less deliverable, more rigid catheter systems, excessive clogging, and excessive blood removal. Also, typical mechanical thrombectomy systems are generally designed to remove soft, fresher clots, and encounter problems when attempting to remove the more prevalent hard, older clots. Moreover, typical mechanical thrombectomy systems have trouble removing blood clots from the vessel wall.

To overcome the technical deficiencies of typical mechanical thrombectomy systems, physicians often perform multiple catheter removals to try to mobilize and aspirate a clot from a patient's venous system. Even then, it may be impossible for any human to fully aspirate a clot using a typical mechanical thrombectomy system. For example, a typical mechanical thrombectomy system may include a component inserted into the venous system of a patient that is actuated to aspirate a clot. However, aspiration of the clot may involve actuating the component five, six, or more times a second. It is impossible for any human to actuate such a component this many times a second, let alone determine how many times a second would be sufficient to aspirate the clot given the unique characteristics of the structure of a patient's venous system, the composition of the patient's blood, the clot to be aspirated, and/or the like. Thus, procedures that use typical mechanical thrombectomy systems produce poor clot aspiration results and can cause significantly increased procedure times.

Accordingly, described herein is an improved mechanical thrombectomy system that uses artificial intelligence to acquire, extrude, segment, and/or aspirate more effectively blood clots regardless of the age, size (e.g., length, diameter, etc.), solidness, or location of the blood clots while lowering the bleeding risk during the operation. For example, the improved mechanical thrombectomy system described herein can include a catheter, one or more sensors coupled to the catheter, and a control unit coupled to the catheter and sensor(s) that uses artificial intelligence to acquire, extrude, segment, and/or aspirate clots.

The catheter may include an outer sheath, a valve, and an inner sheath. The outer and inner sheaths may have cylindric shapes that at least partially traverse the length of the catheter, with the diameter of the inner sheath being smaller than the diameter of the outer sheath. A distal end of the catheter may be inserted into a venous system, and a proximal end of the catheter may be coupled to a collection canister that stores clot pieces aspirated from the venous system. The valve may be positioned at the distal end of the catheter. The inner sheath may move in an axial direction between a closed or retracted position, a flush position, and/or an open position. When in a retracted position, the inner sheath may sit inside the outer sheath and the valve may be closed such that objects outside the distal end of the catheter are prevented from entering the inner sheath. When in a flush position, the inner sheath may sit inside the outer sheath, an end of the inner sheath at the distal end of the catheter may be closer to the valve than when the inner sheath is in the retracted position, and the valve may be closed such that objects outside the distal end of the catheter are prevented from entering the inner sheath. When in an open position, at least a portion of the end of the inner sheath at the distal end of the catheter may be positioned outside the outer sheath and the valve may be open.

The control unit can include a motor and a computing system. The motor may be mechanically coupled to at least the inner sheath and/or the outer sheath and can actuate the inner sheath and/or outer sheath, such as by causing the inner sheath to move relative to the outer sheath and between the retracted position, the flush position, and/or the open position. In particular, the motor may move the inner sheath between the retracted position and the open position when the catheter is being used to acquire, extrude, segment, and/or aspirate a clot. The motor may move the inner sheath between the retracted position, the flush position, and/or the open position when a flushing operation is being implemented to flush clot pieces through the inner sheath into the collection canister and/or to otherwise clean the inner sheath.

The computing system can use artificial intelligence to determine the appropriate position of the inner sheath, such as the distance or amplitude that the inner sheath should be moved to reach the flush or open positions and the frequency at which the inner sheath should be moved between the retracted position and the flush position and/or between the retracted position and the open position. For example, the sensor(s) can include one or more flow sensors (e.g., a sensor that detects a rate at which blood, clots, and/or other objects are being sucked into the inner sheath at the distal end of the catheter), one or more contact sensors (e.g., a sensor that measures a contact pressure between a clot and the inner sheath), one or more temperature sensors (e.g., a thermistor that can be used to measure a temperature at a distal end of the catheter, and ultimately to calculate a velocity of objects flowing into the distal end of the catheter using the measured temperature and/or to calculate a fluid pressure at the distal end of the catheter using the calculated velocity), one or more pressure sensors (e.g., a sensor that measures a fluid pressure at the distal end of the catheter), one or more cameras (e.g., a camera, such as an infrared camera, inserted into the venous system that captures one or more images of a clot in the venous system, which may be optionally located at a distal end of the catheter), one or more sensors that measure the length of clot pieces as the clot pieces are being aspirated into the collection canister and/or the distance between clot pieces as the clot pieces are being aspirated into the collection canister (e.g., a flow sensor that can determine the length of a clot piece and/or distance between clot pieces based on changes in the flow rate of objects flowing through the inner sheath, a camera that can capture one or more images of clot pieces after the clot pieces have exited the venous system and are being aspirated through the inner sheath toward the collection canister, etc.), and/or the like. The sensor(s) can communicate with the computing system (e.g., via a wired or wireless connection) directly and/or indirectly via the motor to provide the computing system with one or more measurements. Similarly, the motor can communicate with the computing system (e.g., via a wired or wireless connection) to provide one or more operational parameters (e.g., current motor power consumption, motor resistance, etc.). The computing system can periodically apply the received sensor measurement(s), the received motor operational parameter(s), an indication of the type of valve at the distal end of the catheter (e.g., duckbill, umbrella, flapper, etc.), and/or an indication of a type of motor present in the control unit (e.g., direct current (DC) motor, alternating current (AC) motor, direct drive motor, linear motor, rotary motor, stepper motor, brushless motor, brushed motor, air-cooled motor, liquid-cooled motor, single-phase motor, two-phase motor, three-phase motor, etc.) as input(s) to an artificial intelligence model (e.g., a machine learning model, a neural network, etc.) that is trained to output an amplitude that defines a distance by which the inner sheath should move from the retracted position to one of the flush position or the open position and a frequency by which the inner sheath should oscillate between the retracted position and the flush or open position. In response to providing the input(s) to the trained artificial intelligence model, the trained artificial intelligence model may output an amplitude (e.g., in millimeters, such as 0.00 mm, 0.05 mm, 1 mm, 2 mm, 3 mm, etc.) and a frequency (e.g., in Hz, such as 0 Hz, 4 Hz, 5 Hz, 6 Hz, etc.). The computing system may then send a signal to the motor that instructs the motor to adjust operation such that the inner sheath oscillates between the retracted position and the flush or open position at the outputted frequency or at about the outputted frequency (e.g., within an error rate of the outputted frequency, where the error rate can be 0.01%, 0.1%, 1%, etc.), where the distance by which the inner sheath moves to reach the flush or open position from the retracted position matches the outputted amplitude or closely matches the outputted amplitude (e.g., within an error rate of the outputted amplitude, where the error rate can be 0.01%, 0.1%, 1%, etc.).

The computing system can apply input(s) to the trained artificial intelligence model one or more times during a procedure. For example, the computing system can apply input(s) to the trained artificial intelligence model automatically every 1 ms, 1 second, 10 seconds, etc., in response to a request from a physician, in response to the power consumption of the motor exceeding a threshold value, initially as the procedure begins, and/or the like. Thus, the computing system may cause the operation of the motor to be adjusted one or more times during a single procedure.

The control unit or a remote computing system can perform the initial training of the artificial intelligence model and/or any re-training or updating of the trained artificial intelligence model. For example, the control unit or remote computing system can train or re-train the artificial intelligence model using training data that includes individual data groups labeled with an amplitude value and a frequency value. Each data group can include one or more sensor measurements (e.g., any of the sensor measurements described herein), one or more motor operational parameters (e.g., any of the motor operational parameters described herein), an indication of the type of valve present at the distal end of the catheter when the sensor measurements were captured, and an indication of the type of motor used to actuate the inner sheath of the catheter. The amplitude value label applied to a data group may represent the amplitude defining the position of the flush or open position that resulted in the best or nearly the best acquisition, extrusion, segmentation, and/or aspiration of a clot and/or the best or nearly the best cleaning of the inner sheath given the sensor measurement(s), motor operational parameter(s), the type of valve, and the type of motor that form the data group. Similarly, the frequency value label applied to a data group may represent the frequency of oscillation of the inner sheath that resulted in the best or nearly the best acquisition, extrusion, segmentation, and/or aspiration of a clot and/or the best or nearly the best cleaning of the inner sheath given the sensor measurement(s), motor operational parameter(s), the type of valve, and the type of motor that form the data group.

The control unit or remote computing system may perform the training or re-training of the artificial intelligence model asynchronously with the use of the improved mechanical thrombectomy system described herein. For example, the control unit or remote computing system can train and/or re-train the artificial intelligence model prior to a time when an individual unit of the improved mechanical thrombectomy system described herein is first used in a procedure such that the trained artificial intelligence model can be stored in the computing system of the individual unit and be available for use when the individual unit is used in a procedure for the first time. In other words, the computing system of an individual unit may be pre-loaded with a trained artificial intelligence model or updated to include a trained artificial intelligence model prior to a first use. Alternatively or in addition, the control unit or remote computing system can train and/or re-train the artificial intelligence model while an individual unit of the improved mechanical thrombectomy system described herein is first used in a procedure or at a time after the individual unit is first used in a procedure. In other words, the computing system of an individual unit can receive a trained or re-trained artificial intelligence model while or after the individual unit has first been used (which may be applicable, for example, in situations in which the control unit of the improved mechanical thrombectomy system is reusable). If the remote computing system performs the training or re-training in any scenario, the remote computing system can transmit the trained artificial intelligence model over a network to the computing system of an individual unit for storage, the remote computing system can export the trained artificial intelligence model to a physical storage medium (e.g., a hard disk, flash memory, a solid state drive, etc.) and the physical storage medium can be coupled to the computing system of the individual unit to transfer the trained artificial intelligence model to the computing system of the individual unit for storage, and/or the like.

While the present disclosure describes the control unit or remote computing system as training a single artificial intelligence model for use in determining the movements of an inner sheath of the catheter, this is not meant to be limiting. The control unit or remote computing system can train multiple artificial intelligence models, where each trained model produces a specific output, is specific to a type of valve, is specific to a type of motor, and/or the like. For example, the control unit or remote computing can train one artificial intelligence model to output an amplitude and train another artificial intelligence model to output a frequency. In this example, the training data used to perform the training may include data groups as described above, but the training data used to train the amplitude-outputting artificial intelligence model may include data groups labeled with amplitude values and the training data used to train the frequency-outputting artificial intelligence model may include data groups labeled with frequency values. As another example, the control unit or remote computing system can train multiple artificial intelligence models, where each is associated with a particular type of valve. In this example, the training data used to perform the training may include data groups that include data gathered from mechanical thrombectomy systems that have the particular type of valve (and therefore the data groups may not include the valve type data and/or the control unit or remote computing system can leave out the valve type data when the training is performed). As another example, the control unit or remote computing system can train multiple artificial intelligence models, where each is associated with a particular type of motor. In this example, the training data used to perform the training may include data groups that include data gathered from mechanical thrombectomy systems that have the particular type of motor (and therefore the data groups may not include the motor type data and/or the control unit or remote computing system can leave out the motor type data when the training is performed).

The control unit or remote computing system can train any combination of artificial intelligence models described herein (e.g., train one artificial intelligence model that is specific to a first type of valve and outputs an amplitude, train a second artificial intelligence model that is specific to a first type of valve and outputs a frequency, train a third artificial intelligence model that is specific to a second type of valve and outputs an amplitude, train a fourth artificial intelligence model that is specific to a second type of valve and outputs a frequency, etc.; train one artificial intelligence model that is specific to a first type of valve and to a first type of motor, train a second artificial intelligence model that is specific to a first type of valve and to a second type of motor, train a third artificial intelligence model that is specific to a second type of valve and to a first type of motor, etc.; train one artificial intelligence model that is specific to a first type of valve, that is specific to a first type of motor, and outputs an amplitude, train a second artificial intelligence model that is specific to a first type of valve, that is specific to a first type of motor, and outputs a frequency, train a third artificial intelligence model that is specific to a first type of valve, that is specific to a second type of motor, and outputs an amplitude, train a fourth artificial intelligence model that is specific to a first type of valve, that is specific to a second type of motor, and outputs a frequency, etc.; train one artificial intelligence model that is specific to a first type of valve and outputs an amplitude, train a second artificial intelligence model that is specific to a first type of valve and outputs a frequency, train a third artificial intelligence model that is specific to a second type of valve and outputs an amplitude and frequency, etc.; etc.), with the training data being adjusted accordingly in a manner as described herein. The type of artificial intelligence model loaded on or sent to an individual unit of the improved mechanical thrombectomy system described herein may match the characteristics of the individual unit (e.g., if an individual unit includes a first type of valve and a second type of motor, the individual unit may be loaded with and/or receive an artificial intelligence model trained specifically for the first type of valve and/or the second type of motor).

By using artificial intelligence, the improved mechanical thrombectomy system described herein may be able to determine the appropriate number of times per second to actuate the inner sheath or how far to actuate the inner sheath to effectively acquire, extrude, segment, and/or aspirate a blood clot. As described above, it may be impossible for a human to make this determination manually because the determination may depend on the unique characteristics of the structure of a patient's venous system, the composition of the patient's blood, the clot to be aspirated, and/or the like, and no human has the ability to observe or identify these unique characteristics while a procedure is taking place. In fact, even if a human knew how many times per second the inner sheath should be actuated, the number of times per second is generally high enough (e.g., 4 times per second, 5 times per second, 6 times per second, etc.) to make it impossible for any human to physically actuate the inner sheath that number of times per second. While a motor may be able to actuate the inner sheath the appropriate number of times per second and/or a desired distance, even a motor may have trouble maintaining the amplitude and/or frequency due to variations in the structure of a patient's venous system, the composition of the patient's blood, the size, age, and/or composition of the clot to be aspirated, and/or the like. The trained artificial intelligence model, however, may take some or all of these characteristics into account when outputting an amplitude and frequency. In some cases, the amplitude and/or frequency outputted by the artificial intelligence model may be lower or higher than the actual amplitude and/or actual frequency that would best acquire, extrude, segment, and/or aspirate a blood clot. Specifically, if the motor was instructed to actuate the inner sheath at the actual amplitude and/or actual frequency, the motor may not be able to do so given resistance produced by the structure of a patient's venous system, the composition of the patient's blood, the size, age, and/or composition of the clot to be aspirated, and/or the like. The amplitude and/or frequency outputted by the trained artificial intelligence model, however, may be at a level that causes the motor to actuate the inner sheath at the actual amplitude and/or actual frequency. Thus, use of artificial intelligence may improve the functionality of the motor as well.

The foregoing aspects and many of the attendant advantages of this disclosure will become more readily appreciated as the same become better understood by reference to the following detailed description, when taken in conjunction with the accompanying drawings.

### Example Artificial Intelligence—Based Mechanical Thrombectomy System Environment

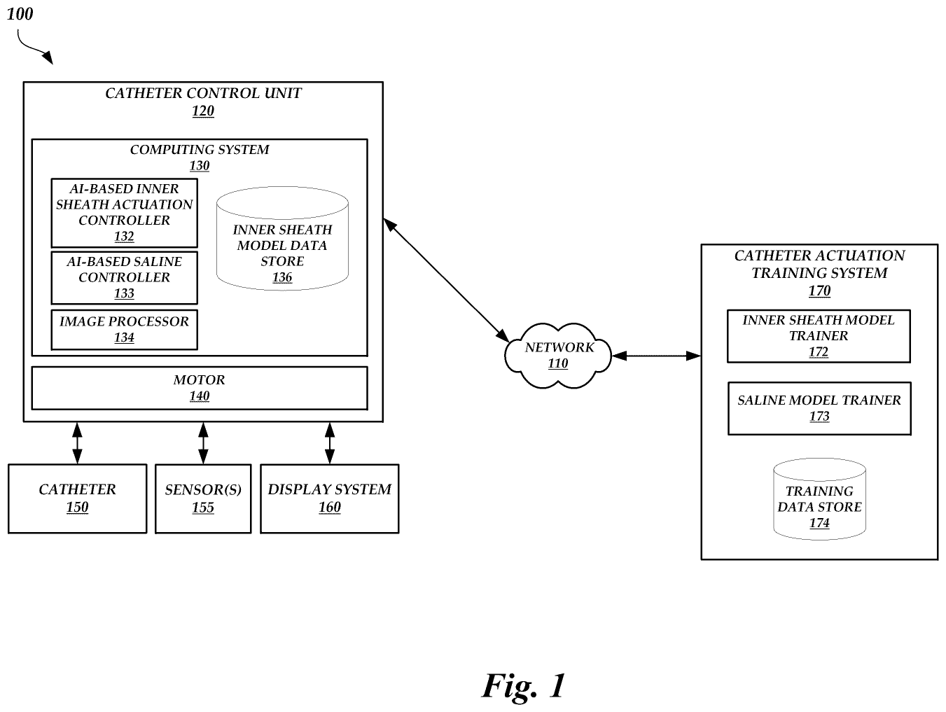

FIG. 1 is a block diagram of an illustrative operating environment 100 of a mechanical thrombectomy system in which a catheter control unit 120 uses artificial intelligence to cause actuation of a catheter 150. The operating environment 100 further includes one or more sensors 155 and/or a display system 160 in communication with the catheter control unit 120 and a catheter actuation training system 170 that may communicate with the catheter control unit 120 via a network 110.

The catheter control unit 120 can include a computing system 130 and a motor 140. The computing system 130 can be configured to determine an amplitude and/or frequency by which an inner sheath of the catheter 150 should move during a procedure. The computing system 130 may be a single computing device, or it may include multiple distinct computing devices. The components of the computing system 130 can each be implemented in application-specific hardware (e.g., one or more application-specific integrated circuits (ASICs)) such that no software is necessary, or as a combination of hardware and software (e.g., a single-board microcontroller, a multi-board microcontroller, etc. that may include one or more microprocessors, memory (e.g., flash memory), one or more input/output (I/O) pins, one or more transceivers, one or more printed circuit expansion boards, and/or like hardware and that can be programmed using firmware or software). In some embodiments, the computing system 130 may include additional or fewer components than illustrated in FIG. 1.

The motor 140 may be coupled to the computing system 130 via a wired or wireless connection. The motor 140 may be configured to transmit operational parameters to the computing system 130 via the wired or wireless connection. The operational parameters can include current motor power consumption, motor resistance, and/or the like. The motor 140 may also be coupled to at least an inner sheath of the catheter 150. The motor 140 can be configured to actuate the inner sheath and/or outer sheath, such as by causing the inner sheath to move relative to the outer sheath and between the retracted position, the flush position, and/or the open position. In particular, the motor 140 may move the inner sheath between the retracted position and the open position when the catheter is being used to acquire, extrude, segment, and/or aspirate a clot. The motor 140 may move the inner sheath between the retracted position, the flush position, and/or the open position and/or keep the inner sheath in the retracted position or the flush position when a flushing operation is being implemented to flush clot pieces through the inner sheath into a collection canister coupled to the catheter control unit 120 and/or to otherwise clean the inner sheath. The motor 140 may further be coupled a power source (e.g., a battery, an AC power supply, a DC power supply, etc.) that supplies power to facilitate motor operation.

The catheter 150 may include an outer sheath, a valve, and an inner sheath. The outer and inner sheaths may have cylindric shapes that at least partially traverse the length of the catheter 150, with the diameter of the inner sheath being smaller than the diameter of the outer sheath. A distal end of the catheter 150 may be inserted into a venous system, and a proximal end of the catheter may be coupled to a collection canister that stores clot pieces aspirated from the venous system. The valve may be positioned at the distal end of the catheter 150. The inner sheath may move in an axial direction between a closed or retracted position, a flush position, and/or an open position. When in a retracted position, the inner sheath may sit inside the outer sheath and the valve may be closed such that objects outside the distal end of the catheter 150 are prevented from entering the inner sheath. When in a flush position, the inner sheath may sit inside the outer sheath, an end of the inner sheath at the distal end of the catheter 150 may be closer to the valve than when the inner sheath is in the retracted position, and the valve may be closed such that objects outside the distal end of the catheter 150 are prevented from entering the inner sheath. The inner sheath may be in a flush position to help clear debris from the inner sheath. As described in greater detail below, irrigation fluid (e.g., saline) can be injected into the catheter 150 between the inner and outer sheaths and sent towards a distal end of the catheter 150. The valve at the distal end of the catheter 150, when closed, may prevent some or all of the saline from leaving the catheter 150, and a vacuum that pulls objects from the distal end of the catheter 150 to the proximal end of the catheter 150 within the inner sheath may pull the saline through the inner sheath as well. Pulling the saline through the inner sheath from the distal end of the catheter 150 to the proximal end of the catheter 150 may help flush and clear any clot pieces or other debris that are lodged in the inner sheath. Based on sensor 155 measurements, the trained artificial intelligence model may determine that the inner sheath is sufficiently clogged and output an amplitude that results in the inner sheath being actuated to a flush position (rather than an open position). When in an open position, at least a portion of the end of the inner sheath at the distal end of the catheter 150 may be positioned outside the outer sheath and the valve may be open.

The one or more sensors 155 can include one or more flow sensors (e.g., a sensor that detects a rate at which blood, clots, and/or other objects are being sucked into the inner sheath at the distal end of the catheter 150) located internal to the catheter 150, external to the catheter 150, in a collection canister, etc., one or more contact sensors (e.g., a sensor that measures a contact pressure between a clot and the inner sheath), one or more temperature sensors (e.g., a thermistor that can be used to measure a temperature at a distal end of the catheter 150, and ultimately to calculate a velocity of objects flowing into the distal end of the catheter 150 using the measured temperature and/or to calculate a fluid pressure at the distal end of the catheter 150 using the calculated velocity), one or more pressure sensors (e.g., a sensor that measures a fluid pressure at the distal end of the catheter 150), one or more cameras (e.g., a camera, such as an infrared camera or other infrared optical sensor, inserted into the venous system that captures one or more images of a clot in the venous system, which may be optionally located at a distal end of the catheter 150), one or more sensors that measure the length of clot pieces as the clot pieces are being aspirated into the collection canister and/or the distance between clot pieces as the clot pieces are being aspirated into the collection canister (e.g., a flow sensor that can determine the length of a clot piece and/or distance between clot pieces based on changes in the flow rate of objects flowing through the inner sheath, a camera that can capture one or more images of clot pieces after the clot pieces have exited the venous system and are being aspirated through the inner sheath toward the collection canister, etc.), one or more sensors that measures impedance (e.g., a sensor that measures impedance at a distal end of the catheter 150), and/or the like. Some or all of the sensors 155 can be coupled to a distal end of the catheter 150 (e.g., coupled to an outer sheath of the catheter 150, coupled between the inner and outer sheaths of the catheter 150, coupled inside the inner sheath of the catheter 150). Other sensors 155 may be coupled to or near a proximal end of the catheter 150 or to the catheter control unit 120 near a proximal end of the catheter 150 (e.g., one or more sensors that measure the length of clot pieces as the clot pieces are being aspirated into the collection canister and/or the distance between clot pieces as the clot pieces are being aspirated into the collection canister). Regardless of the location of the sensor(s) 155, the sensor(s) 155 can communicate with the computing system (e.g., via a wired or wireless connection) directly and/or indirectly via the motor 140 to provide the computing system 130 with one or more sensor 155 measurements.

The computing system 130 may include various modules, components, data stores, and/or the like to provide the artificial intelligence functionality described herein. For example, the computing system 130 may include an artificial intelligence-based inner sheath actuation controller 132, an artificial intelligence-based saline controller 133, an image processor 134, and an inner sheath model data store 136.

The artificial intelligence-based inner sheath actuation controller 132 can use one or more trained artificial intelligence models to determine an amplitude and/or frequency by which the inner sheath of the catheter 150 should move during a procedure. For example, the artificial intelligence-based inner sheath actuation controller 132 can obtain sensor 155 measurement(s) from the sensor(s) 155 and/or motor 140 operational parameters from the motor 140. The artificial intelligence-based inner sheath actuation controller 132 can also obtain a trained artificial intelligence model from the inner sheath model data store 136, where the trained artificial intelligence model is trained to output an amplitude that defines a distance by which the inner sheath should move from the retracted position to one of the flush position or the open position and a frequency by which the inner sheath should oscillate between the retracted position and the flush or open position. The artificial intelligence-based inner sheath actuation controller 132 can periodically apply the received sensor 155 measurement(s), expected sensor 155 measurement(s) (e.g., expected flow rate through the inner sheath), the received motor 140 operational parameter(s), an indication of the type of valve at the distal end of the catheter 150 (e.g., duckbill, umbrella, flapper, etc.), and/or an indication of a type of motor 140 present in the catheter control unit 120 (e.g., DC motor, AC motor, direct drive motor, linear motor, rotary motor, stepper motor, brushless motor, brushed motor, air-cooled motor, liquid-cooled motor, single-phase motor, two-phase motor, three-phase motor, etc.) as input(s) to the trained artificial intelligence model. In response to the artificial intelligence-based inner sheath actuation controller 132 providing the input(s) to the trained artificial intelligence model, the trained artificial intelligence model may output an amplitude (e.g., in millimeters, such as 0.00 mm, 0.05 mm, 1 mm, 2 mm, 3 mm, etc.) and/or a frequency (e.g., in Hz, such as 0 Hz, 4 Hz, 5 Hz, 6 Hz, etc.).

As described herein, the trained artificial intelligence model may be a general model applicable to a catheter 150 with any type of valve and/or to a catheter control unit 120 with any type of motor 140. Alternatively, the trained artificial intelligence model may be specific to a type of valve and/or to a type of motor 140. If the trained artificial intelligence model is specific to a type of valve, then the artificial intelligence-based inner sheath actuation controller 132 may not provide an indication of the type of valve as an input to the trained artificial intelligence model. Similarly, if the trained artificial intelligence model is specific to a type of motor 140, then the artificial intelligence-based inner sheath actuation controller 132 may not provide an indication of the type of motor 140 as an input to the trained artificial intelligence model. In addition, the artificial intelligence-based inner sheath actuation controller 132 may retrieve and use multiple trained artificial intelligence models (e.g., one for amplitude and one for frequency, where each model receives the same input(s)) to produce the desired outputs.

Optionally, the image processor 134 may process some or all of the sensor 155 measurements in a manner as described below. The artificial intelligence-based inner sheath actuation controller 132 may provide the processed sensor 155 measurement(s) rather than the raw sensor 155 measurement(s) as input(s) to the trained artificial intelligence model.

Once the trained artificial intelligence model(s) output the amplitude and frequency, the artificial intelligence-based inner sheath actuation controller 132 may send a signal to the motor 140 that instructs the motor 140 to adjust operation (e.g., actuation of the inner sheath) such that the inner sheath oscillates between the retracted position and the flush or open position at the outputted frequency or at about the outputted frequency (e.g., within an error rate of the outputted frequency, where the error rate can be 0.01%, 0.1%, 1%, etc.), where the distance by which the inner sheath moves to reach the flush or open position from the retracted position matches the outputted amplitude or closely matches the outputted amplitude (e.g., within an error rate of the outputted amplitude, where the error rate can be 0.01%, 0.1%, 1%, etc.). If the outputted amplitude is at least equal to or greater than a distance between a distal end of the inner sheath while in the retracted position and a distal end of the outer sheath, then this may result in the inner sheath being moved to an open position. On the other hand, if the outputted amplitude is less than a distance between a distal end of the inner sheath while in the retracted position and a distal end of the outer sheath, then this may result in the inner sheath being moved to a flush position. In some instances, the outputted amplitude may be a value (e.g., 0.00 mm, 0.01 mm, 0.02 mm, etc.) such that the inner sheath remains in the retracted position, moves slowly (e.g., at a low outputted frequency, such as 0 Hz, 0.5 Hz, 0.8 Hz, 1 Hz, etc.) from the retracted position toward a position between the retracted position and the flush position, remains in the flush position, and/or moves slowly (e.g., at a low outputted frequency, such as 0 Hz, 0.5 Hz, 0.8 Hz, 1 Hz, etc.) from the flush position toward a position between the retracted position and the flush position. The trained artificial intelligence model may output such an amplitude if, for example, the measured blood loss through the inner sheath (e.g., as determined by the flow rate measured by a flow sensor 155, where a higher flow rate indicates increased blood loss; as determined by an impedance measured by an impedance sensor 155, where a higher impedance may indicate increased cell volume and therefore increased blood loss; as determined by an output from an infrared optical sensor 155 that may detect increased blood flow and therefore increased blood loss) is greater than a threshold amount and/or if the flow rate through the inner sheath changes from an expected flow rate through the inner sheath (e.g., changes to a flow rate value that is higher than the expected flow rate value).

The artificial intelligence-based inner sheath actuation controller 132 can apply input(s) to the trained artificial intelligence model one or more times during a procedure. For example, the artificial intelligence-based inner sheath actuation controller 132 can apply input(s) to the trained artificial intelligence model automatically every 1 ms, 1 second, 10 seconds, etc., in response to a request from a physician, in response to the power consumption of the motor 140 exceeding a threshold value, initially as the procedure begins, and/or the like. Thus, the artificial intelligence-based inner sheath actuation controller 132 may cause the operation of the motor 140 to be adjusted one or more times during a single procedure.

The artificial intelligence-based saline controller 133 may drive a component that controls the saline flow pressure or flow rate through the inner sheath (e.g., a saline pump) using a trained saline artificial intelligence model. As described herein, saline can be injected into the catheter 150 between the inner and outer sheaths and sent towards a distal end of the catheter 150. The valve at the distal end of the catheter 150, when closed, may prevent some or all of the saline from leaving the catheter 150, and a vacuum that pulls objects from the distal end of the catheter 150 to the proximal end of the catheter 150 within the inner sheath may pull the saline through the inner sheath as well. Pulling the saline through the inner sheath from the distal end of the catheter 150 to the proximal end of the catheter 150 may help flush and clear any clot pieces or other debris that are lodged in the inner sheath. In some cases, a flow sensor 155 (e.g., a flow sensor that detects a rate at which blood, clots, and/or other objects are being sucked into the inner sheath at the distal end of the catheter 150) may indicate a flow rate of objects being sucked into the inner sheath at the distal end of the catheter 150 has a value that is below a threshold flow rate value. The flow rate having a value below the threshold flow rate value may indicate that the inner sheath is still clogged with one or more objects and/or the rate at which the clog is being removed is lower than expected. Adjusting the flow rate of the saline (e.g., increasing the flow rate of the saline, decreasing the flow rate of the saline, etc.) may improve the flushing or clearing of the inner sheath. The artificial intelligence-based saline controller 133 can obtain a trained saline artificial intelligence model and apply one or more flow rate measurements obtained from the flow sensor 155, a current saline flow pressure or flow rate value (e.g., from a flow sensor 155 that measures saline flow or from the component (e.g., a saline pump, which may be internal or external to the catheter control unit 120) that controls saline flow), one or more operational parameters of the component that controls saline flow pressure or flow rate, an indication of the type of valve present at the distal end of the catheter 150 when the flow sensor 155 measurements were captured, and/or an indication of the type of component used to control saline flow pressure or flow rate (e.g., type of saline pump) as an input to the trained saline artificial intelligence model. As a result, the trained saline artificial intelligence model may output a saline flow pressure or flow rate or an indication of an adjustment to be made to a current saline flow pressure or flow rate. The artificial intelligence-based saline controller 133 can then send a signal to the component that controls the saline flow pressure or flow rate (e.g., to the saline pump) that instructs the component to adjust the saline flow pressure or flow rate to the saline flow pressure or flow rate output by the trained saline artificial intelligence model (e.g., if the model outputs a new saline flow pressure or flow rate value) or by an amount indicated by the output of the trained saline artificial intelligence model (e.g., if the model outputs an indication of an adjustment to be made).

The artificial intelligence-based saline controller 133 can apply input(s) to the trained saline artificial intelligence model one or more times during a procedure. For example, the artificial intelligence-based saline controller 133 can apply input(s) to the trained saline artificial intelligence model automatically every 1 ms, 1 second, 10 seconds, etc., in response to a request from a physician, in response to the flow rate through the inner sheath falling below a threshold value, initially as the procedure begins, and/or the like. Thus, the artificial intelligence-based saline controller 133 may cause the saline flow pressure or flow rate to be adjusted one or more times during a single procedure.

The image processor 134 can be configured to process one or more images captured by a sensor 155, such as a camera coupled to a distal end of the catheter 150 and/or a camera positioned to capture the length of clot pieces being aspirated toward the collection canister and/or the distance between clot pieces being aspirated into the collection canister. For example, a camera coupled to the distal end of the catheter 150 may capture one or more images of a boundary of a vessel and/or a boundary of a clot in the vessel. The image processor 134 can use image processing techniques (e.g., by performing edge detection, by identifying differences in temperature (e.g., if an infrared image), etc.) to determine, for example, a size of the clot. The image processor 134 can cause the display system 160 to display one or more of the captured images with an indication of the determined clot size, can provide the determined clot size to the artificial intelligence-based inner sheath actuation controller 132 for use as an input to the trained artificial intelligence model, and/or can cause an indicator light present on the catheter control unit 120 or the catheter 150 to turn on if the image processor 134 determines that the determined clot size is less than a threshold size (where the indicator light, when on, may inform a physician that a sufficient amount of the clot has been aspirated and the catheter 150 can be removed).

As another example, a camera positioned to capture the length of clot pieces being aspirated toward the collection canister and/or the distance between clot pieces can capture corresponding image(s) and provide the image(s) to the image processor 134. The image processor 134 can use image processing techniques (e.g., by performing edge detection based on differences in the color of a clot piece and other material being aspirated within the inner sheath toward the collection canister) to estimate a size of one or more clot pieces as the clot pieces are being aspirated toward the collection canister and/or a distance between clot pieces. The image processor 134 can provide the determined clot piece length(s) and/or the distance(s) between clot pieces to the artificial intelligence-based inner sheath actuation controller 132 for use as input(s) to the trained artificial intelligence model.

The inner sheath model data store 136 may store one or more trained artificial intelligence models (e.g., a machine learning model, a neural network, etc.) that are each trained to output an amplitude and/or a frequency. As described herein, a trained artificial intelligence model stored in the inner sheath model data store 136 can be a general artificial intelligence model applicable to all types of catheters 150 or may be an artificial intelligence model specific to a type of valve at the distal end of the catheter 150 and/or specific to a type of motor 140 present in the catheter control unit 120. Optionally, the inner sheath model data store 136 may also store one or more trained saline artificial intelligence models (e.g., a machine learning model, a neural network, etc.) that are each trained to output a new saline flow pressure or flow rate or an adjustment to an existing saline flow pressure or flow rate. While the inner sheath model data store 136 is depicted as being internal to the computing system 130, this is not meant to be limiting. For example, not shown, the inner sheath model data store 136 can be located external to the computing system 130.

The display system 160 may include one or more displays (e.g., an organic light-emitting diode (OLED) display, a light-emitting diode (LED) display, a liquid-crystal display (LCD), a mobile phone screen, a tablet screen, a laptop screen, a workstation screen, etc.) configured to display information obtained from the catheter control unit 120, the catheter 150, and/or the sensor(s) 155 (e.g., images captured by a camera at a distal end of the catheter 150). The display system 160 may be positioned, for example, bedside during a procedure. The display system 160 may be in wired or wireless communication with the catheter control unit 120, the catheter 150, and/or the sensor(s) 155.

The catheter actuation training system 170 may be a single computing device, or it may include multiple distinct computing devices, such as computer servers, logically or physically grouped together to collectively operate as a server system. The components of the catheter actuation training system 170 can each be implemented in application-specific hardware (e.g., a server computing device with one or more ASICs) such that no software is necessary, or as a combination of hardware and software. In addition, the modules and components of the catheter actuation training system 170 can be combined on one server computing device or separated individually or into groups on several server computing devices. In some embodiments, the catheter actuation training system 170 may include additional or fewer components than illustrated in FIG. 1.

In some embodiments, the features and services provided by the catheter actuation training system 170 may be implemented as web services consumable via the communication network 110. In further embodiments, the catheter actuation training system 170 is provided by one more virtual machines implemented in a hosted computing environment. The hosted computing environment may include one or more rapidly provisioned and released computing resources, which computing resources may include computing, networking and/or storage devices. A hosted computing environment may also be referred to as a cloud computing environment.

The catheter actuation training system 170 may include various modules, components, data stores, and/or the like to provide the model training functionality described herein. For example, the catheter actuation training system 170 may include an inner sheath model trainer 172, a saline model trainer 173, and a training data store 174.

The inner sheath model trainer 172 can perform the initial training of the artificial intelligence model and/or any re-training or updating of the trained artificial intelligence model. For example, the inner sheath model trainer 172 can train or re-train the artificial intelligence model using training data obtained from the training data store 174 that includes individual data groups labeled with an amplitude value and a frequency value. Each data group can include one or more sensor 155 measurements (e.g., any of the sensor 155 measurements described herein, where such measurements can be actual and/or expected), one or more motor 140 operational parameters (e.g., any of the motor 140 operational parameters described herein), an indication of the type of valve present at the distal end of the catheter 150 when the sensor 155 measurements were captured, and an indication of the type of motor 140 used to actuate the inner sheath of the catheter 150. The amplitude value label applied to a data group may represent the amplitude defining the position of the flush or open position that resulted in the best or nearly the best acquisition, extrusion, segmentation, and/or aspiration of a clot and/or the best or nearly the best cleaning of the inner sheath given the sensor 155 measurement(s), motor 140 operational parameter(s), the type of valve, and the type of motor 140 that form the data group. Similarly, the frequency value label applied to a data group may represent the frequency of oscillation of the inner sheath that resulted in the best or nearly the best acquisition, extrusion, segmentation, and/or aspiration of a clot and/or the best or nearly the best cleaning of the inner sheath given the sensor 155 measurement(s), motor 140 operational parameter(s), the type of valve, and the type of motor 140 that form the data group.

The inner sheath model trainer 172 may perform the training or re-training of the artificial intelligence model asynchronously with the use of the catheter control unit 120 and/or the catheter 150. For example, the inner sheath model trainer 172 can train and/or re-train the artificial intelligence model prior to a time when an individual unit of the catheter control unit 120 and/or catheter 150 is first used in a procedure such that the trained artificial intelligence model can be stored in the computing system 130 of the individual unit and be available for use when the individual unit is used in a procedure for the first time. In other words, the computing system 130 of an individual unit may be pre-loaded with a trained artificial intelligence model or updated to include a trained artificial intelligence model prior to a first use. Alternatively or in addition, the inner sheath model trainer 172 can train and/or re-train the artificial intelligence model while an individual unit of the catheter control unit 120 and/or catheter 150 is first used in a procedure or at a time after the individual unit is first used in a procedure. In other words, the computing system 130 of an individual unit can receive a trained or re-trained artificial intelligence model while or after the individual unit has first been used (which may be applicable, for example, in situations in which the catheter control unit 120 is reusable). The inner sheath model trainer 172 can transmit the trained artificial intelligence model over the network 110 to the computing system 130 of an individual unit for storage, the inner sheath model trainer 172 can export the trained artificial intelligence model to a physical storage medium (e.g., a hard disk, flash memory, a solid state drive, etc.) and the physical storage medium can be coupled to the computing system 130 of the individual unit to transfer the trained artificial intelligence model to the computing system 130 of the individual unit for storage, and/or the like.

The saline model trainer 173 can perform the initial training of the saline artificial intelligence model and/or any re-training or updating of the trained saline artificial intelligence model. For example, the saline model trainer 173 can train or re-train the artificial intelligence model using training data obtained from the training data store 174 that includes individual data groups labeled with an indication of whether a clog is present in the inner sheath. Each data group can include one or more sensor 155 measurements (e.g., any of the sensor 155 measurements described herein, where such measurements can be actual and/or expected), one or more operational parameters of the component that controls saline flow pressure or flow rate (e.g., a saline pump), an indication of the type of valve present at the distal end of the catheter 150 when the sensor 155 measurements were captured, and/or an indication of the type of component used to control saline flow pressure or flow rate (e.g., a type of saline pump).

The saline model trainer 173 may perform the training or re-training of the saline artificial intelligence model asynchronously with the use of the catheter control unit 120 and/or the catheter 150. For example, the saline model trainer 173 can train and/or re-train the saline artificial intelligence model prior to a time when an individual unit of the catheter control unit 120 and/or catheter 150 is first used in a procedure such that the trained saline artificial intelligence model can be stored in the computing system 130 of the individual unit and be available for use when the individual unit is used in a procedure for the first time. In other words, the computing system 130 of an individual unit may be pre-loaded with a trained saline artificial intelligence model or updated to include a trained saline artificial intelligence model prior to a first use. Alternatively or in addition, the saline model trainer 173 can train and/or re-train the saline artificial intelligence model while an individual unit of the catheter control unit 120 and/or catheter 150 is first used in a procedure or at a time after the individual unit is first used in a procedure. In other words, the computing system 130 of an individual unit can receive a trained or re-trained saline artificial intelligence model while or after the individual unit has first been used (which may be applicable, for example, in situations in which the catheter control unit 120 is reusable). The saline model trainer 173 can transmit the trained saline artificial intelligence model over the network 110 to the computing system 130 of an individual unit for storage, the saline model trainer 173 can export the trained saline artificial intelligence model to a physical storage medium (e.g., a hard disk, flash memory, a solid state drive, etc.) and the physical storage medium can be coupled to the computing system 130 of the individual unit to transfer the trained saline artificial intelligence model to the computing system 130 of the individual unit for storage, and/or the like.

The training data store 174 can store training data for use in training one or more artificial intelligence models and/or one or more saline artificial intelligence models. For example, the training data can include sensor 155 measurements and/or motor 140 operational parameters captured during a bench test, by an individual unit of a catheter control unit 120 and/or catheter 150, and/or the like. Alternatively or in addition, the training data can include sensor 155 measurements (e.g., measurements from a flow sensor that measures the flow rate through the inner sheath, measurements from a flow sensor that measures the flow rate of saline, etc.) and/or operational parameters of the component that controls saline flow pressure or flow rate (e.g., operational parameters of a saline pump). While the training data store 174 is depicted as being internal to the catheter actuation training system 170, this is not meant to be limiting. For example, not shown, the training data store 174 can be located external to the catheter actuation training system 170.

While the catheter actuation training system 170 is described herein as performing the artificial intelligence model training, this is not meant to be limiting. Some or all of the functionality described herein as being performed by the catheter actuation training system 170 can be performed by the computing system 130.

### Example Block Diagram for Determining Catheter Actuation

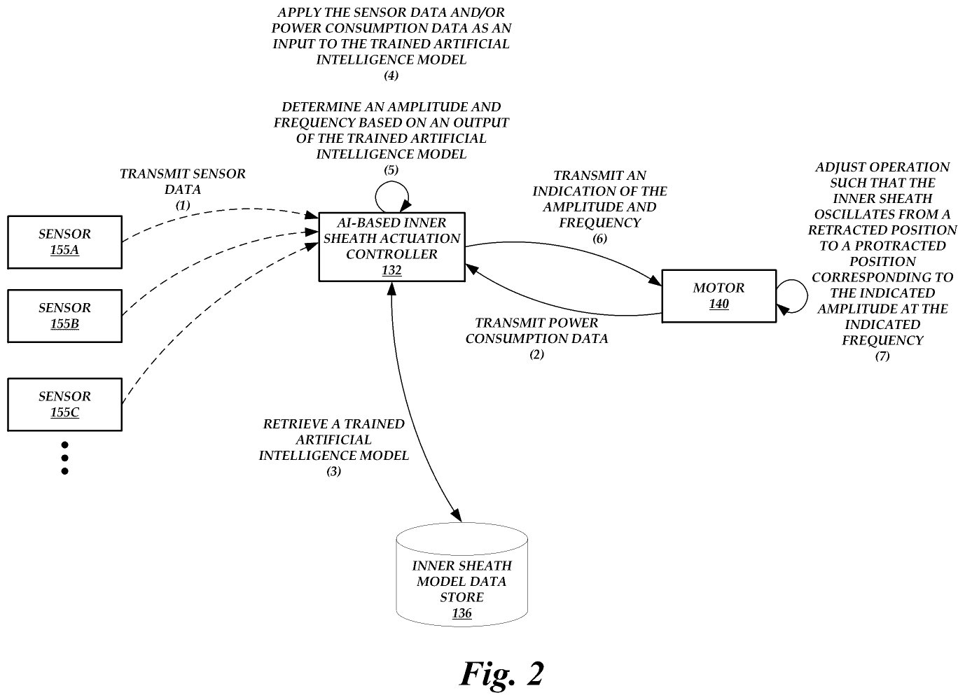

FIG. 2 is a flow diagram illustrating the operations performed by the components of the operating environment 100 of FIG. 1 to determine an amplitude and frequency by which the inner sheath of the catheter 150 is to be actuated. As illustrated in FIG. 2, one or more sensors 155A-C and/or other sensor(s) 155 transmit sensor data to the artificial intelligence-based inner sheath actuation controller 132 at (1). The sensor data can include any of the sensor 155 measurements described herein. The sensor(s) 155A-C and/or other sensor(s) 155 can transmit the sensor data via a wired or wireless connection that optionally passes through the motor 140. Before, during, and/or after the sensor(s) 155A-C and/or other sensor(s) 155 transmit the sensor data, the motor 140 can transmit power consumption data to the artificial intelligence-based inner sheath actuation controller 132. Optionally, the motor 140 can send to the artificial intelligence-based inner sheath actuation controller 132 other operational parameters, such as motor 140 resistance.

The artificial intelligence-based inner sheath actuation controller 132 can retrieve a trained artificial intelligence model from the inner sheath model data store 136 at (3). As described herein, the trained artificial intelligence model may be a general model trained to output an amplitude and/or frequency for a catheter 150 with any type of valve and for any type of motor 140 present in the catheter control unit 120. Alternatively, the trained artificial intelligence model may be specific to a type of valve present in the catheter 150 to be actuated and/or specific to a type of motor 140 present in the catheter control unit 120 coupled to the catheter 150 to be actuated. In some embodiments, the artificial intelligence-based inner sheath actuation controller 132 can retrieve multiple trained artificial intelligence models. For example, one trained artificial intelligence model may be trained to output an amplitude and another trained artificial intelligence model may be trained to output a frequency.

The artificial intelligence-based inner sheath actuation controller 132 can apply the sensor data and/or power consumption data as an input to the trained artificial intelligence model at (4). In further embodiments, the artificial intelligence-based inner sheath actuation controller 132 can apply an indication of the type of valve present in the catheter 150 and/or the type of motor 140 present in the catheter control unit 120 as inputs to the trained artificial intelligence model as well. Based on providing one or more inputs to the trained artificial intelligence model and causing the trained artificial intelligence model to produce an output, the artificial intelligence-based inner sheath actuation controller 132 can determine an amplitude and frequency at (5). The artificial intelligence-based inner sheath actuation controller 132 can transmit an indication of the amplitude and frequency to the motor 140 at (6).

The motor 140 can adjust its operation in response to receiving the indication from the artificial intelligence-based inner sheath actuation controller 132. For example, the motor 140 can adjust operation such that the inner sheath oscillates from a retracted position to a protracted position (e.g., a flush position or an open position) corresponding to the indicated amplitude at the indicated frequency at (7).

Some or all of the operations disclosed with respect to FIG. 2 may be repeated one or more times during a particular procedure. In addition, while the operations disclosed with respect to FIG. 2 are discussed in a particular order, this is not meant to be limiting as one or more of these operations can be performed in a different order.

### Example Catheter

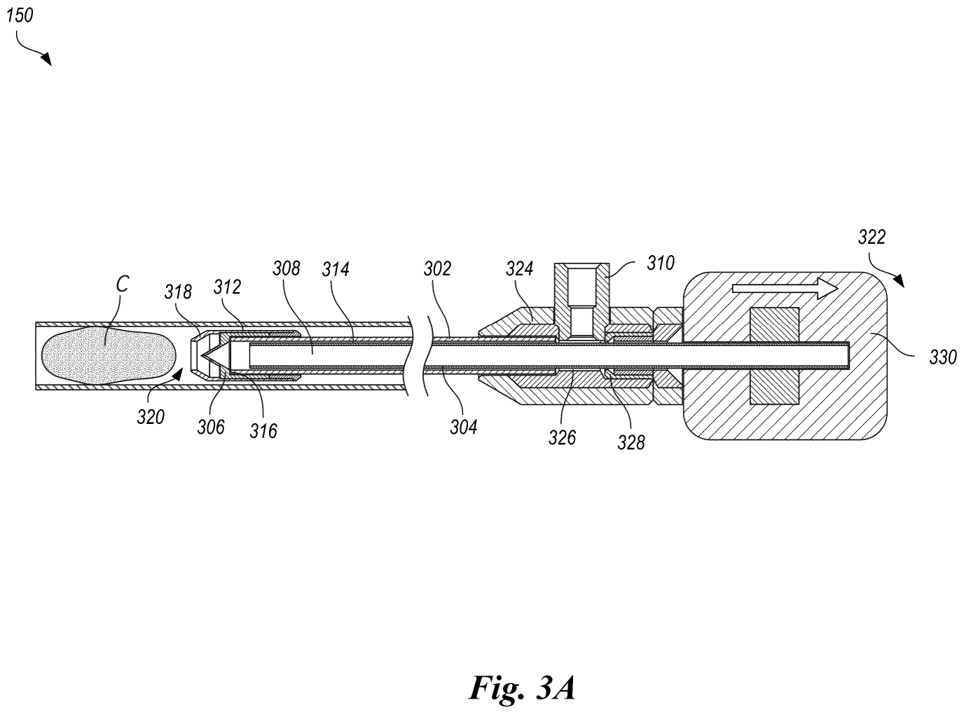

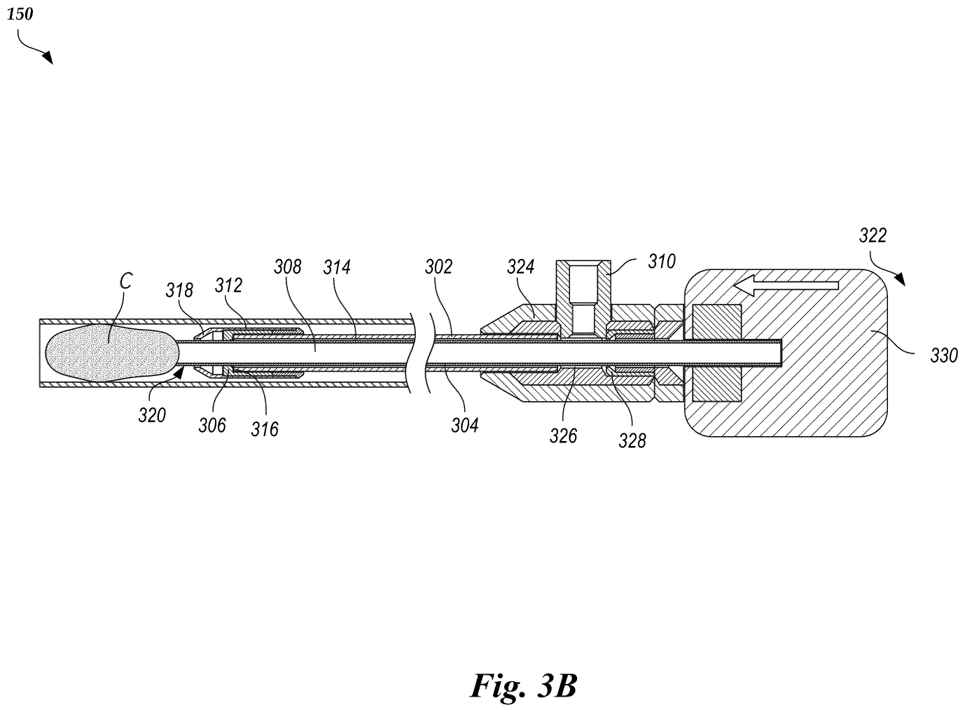

FIGS. 3A-3B illustrate an example catheter 150 in various positions. For example, FIG. 3A illustrates the catheter 150 in a closed or retracted position. As illustrated in FIG. 3A, the catheter 150 includes an outer sheath 302 (also referred to herein as an outer support catheter), an inner sheath 304 (also referred to herein as an inner aspiration catheter), a valve 306, and an inlet 310 in which saline can be injected toward a distal end 320 of the catheter 150. As illustrated, the inner aspiration catheter 304 extends through the outer support catheter 302. The inner sheath 304 may be considered to be in a closed or retracted position because the valve 306 is closed and the inner sheath 304 cannot move any further toward a proximal end 322 of the catheter 150.

As described herein, the saline injected in the inlet 310 may pass through an inlet positioned between the outer sheath 302 and the inner sheath 304 toward the distal end 320 of the catheter 150. Because the inner sheath 304 is in a closed or retracted position and the valve 306 is closed, the force of the vacuum present in the inner sheath 304 when the catheter 150 is in operation may pull the saline from the distal end 320 of the catheter 150 through the inner sheath and toward the proximal end 322 of the catheter 150.

The inner aspiration catheter 304 can define an aspiration lumen 308. The inner aspiration catheter 304 can be operably connected to a vacuum source. The vacuum source can cause the inner aspiration catheter 304 to aspirate clot(s) C through the aspiration lumen 308.

The inner aspiration catheter 304 can include a polymeric material with a reinforcing braid or coil. An outer diameter of the inner aspiration catheter 304 can be at least about 1.0 mm and/or less than or equal to about 12.0 mm, for example between 2.0 mm and 10.0 mm, such as no more than 5.0 mm, no more than 4.0 mm, or no more than 3.0 mm. A wall thickness of the inner aspiration catheter 304 can be less than or equal to about 0.5 mm, less than or equal to about 0.4 mm, less than or equal to about 0.3 mm, less than or equal to about 0.2 mm, or less than or equal to about 0.1 mm.

As shown in FIG. 3A, the aspiration lumen 308 may have a constant diameter. A distal segment of the inner aspiration catheter 304 may include a first material and a proximal segment of the aspiration catheter may include a second material with different properties, e.g., different stiffness than the first material.

In other embodiments, the aspiration lumen 308 may have a variable diameter with a distal portion of the lumen 308 having a smaller diameter than a proximal portion of the lumen 308. The inner aspiration catheter 304 may include a distal segment joined to a proximal segment. The distal segment and the proximal segment may include the same material or different materials. For example, the proximal segment may be stiffer than the distal segment. The distal segment may have a first inner diameter and the proximal segment may have a second inner diameter greater than the first inner diameter. The distal segment may extend into the proximal segment such that an exterior surface of the distal segment is joined to an inner surface of the proximal segment. An outer diameter of the proximal segment may be less than the inner diameter of a valve housing 312. The transition between the distal segment and the proximal segment may form a stop joint that prevents the inner aspiration catheter 304 from moving a certain distance beyond a distal end of the outer support catheter 302. For example, the aspiration catheter may only be able to extend no more than 5 cm (or no more than 3 cm, or no more than 2 cm, no more than 1 cm, no more than 0.5 cm, or no more than 0.1 cm) beyond a distal end of the outer support catheter 302. It is possible however to manually or slowly extend the aspiration tube out of the support tube for a longer distance in a “seeker” mode to complete the procedure if there is a remaining clot in a small distal vessel, to capture this residual clot and rapidly retract it back into the support tube.

The outer support catheter 302 can include an elongate tubular body. The elongate tubular body can include a polymeric material with a reinforcing braid or coil. One or more radiopaque markers may be located along the elongate tubular body.

An inner diameter of the outer support catheter 302 can be greater than an external diameter of the inner aspiration catheter 304 to leave space 314 for fluid to flow between the two catheters. An outer diameter of the outer support catheter 302 can be at least about 1.0 mm and/or less than or equal to about 12.0 mm, for example between 2.0 mm and 10.0 mm or between 3.0 mm and 5.0 mm. An inner diameter of the outer support catheter 302 can be at least 0.1 mm greater than an outer diameter of the inner aspiration catheter 304, for example at least about 0.1 mm greater than the outer diameter of the inner aspiration catheter 304 and/or no more than about 1.0 mm greater the outer diameter of the inner aspiration catheter 304, for example between about 0.25 mm and about 0.75 mm greater than the outer diameter of the inner aspiration catheter 304. A wall thickness of the outer support catheter 302 can be less than or equal to about 0.5 mm, less than or equal to about 0.4 mm, less than or equal to about 0.3 mm, less than or equal to about 0.2 mm, or less than or equal to about 0.1 mm.

The outer support catheter 302 can include a valve 306 at or near a distal end 316 of the elongate tubular body. For example, the valve 306 can be within 15 cm (or within 10 cm, or within 5 cm, within 1 cm, within 0.5 cm, or within 0.1 cm) of the distal end 316 of the elongate tubular body. The valve 306 may be disposed within a lumen of the outer support catheter 302 or external of the outer support catheter 302.

As illustrated, the valve 306 can be secured to the elongate tubular body with a valve housing 312. The valve housing 312 can protect the vessel wall from the valve 306. The valve 306 may be disposed within the valve housing 312 with the valve housing 312 extending distally and/or proximally of the valve 306. The valve 306 may be mechanically or chemically secured within the valve housing 312.

The valve housing 312 can be secured to a distal end 316 of the elongate tubular body, for example by welding or bonding. The valve housing 312 may be secured to an exterior surface of the elongate tubular body. The valve housing 312 can be made of plastic or metal.

An inner diameter of the valve housing 312 may be larger than an inner diameter of the elongate tubular body. But in other arrangements, the valve housing 312 may extend into the elongate tubular body. A diameter of the opening at the distal tip 318 may be less than or equal to the inner diameter of the distal end 316 of the elongate tubular body. The distal tip 318 of the valve housing 312 may form the distal tip of the outer support catheter 302. The distal tip 318 of the valve housing 312 may be tapered. The tapered distal tip 318 can function as a breaking shoulder to disrupt or segment the clot. This helps segment harder clots.

When assembled, the distal end 316 of the elongate tubular body may abut a proximal side of the valve 306 to maintain a position of the valve 306 within the valve housing 312. The distal end 316 of the elongate tubular body may be spaced apart from a proximal facing surface of the valve 306 to allow irrigation fluid to flow into the inner aspiration catheter 304.

The valve housing 312 is optional. The valve 306 may be incorporated directly onto or into the elongate tubular body. For example, a metal ring may be placed within the valve 306 and welded to the reinforcement structure within the elongate tubular body.

The valve 306 can be a one-way valve. As illustrated, the valve 306 is a duckbill valve but may include any of the valve features described above. The valve 306 could be any valve with an opening edge sufficiently rigid to segment a clot. The valve 306 could be any valve that allows the inner aspiration catheter 304 to be advanced and retracted through the valve 306. For example, the valve 306 could be a slit valve or a valve with overlapping leaflets having edges suitable for segmenting the valve. An inner diameter of the valve 306 can be less than an inner diameter of the outer support catheter 302 but greater than an outer diameter of the inner aspiration catheter 304.

The catheter 150 may include a manifold 324 at the proximal end of the outer support catheter 302. The manifold 324 may include an inlet 310 for connection to an irrigation source. The manifold 324 allows irrigation fluid to flow in the space 314 between the outer support catheter 302 and the inner aspiration catheter 304. The manifold 324 also includes a passage 326 through which the inner aspiration catheter 304 extends for connection to a vacuum source.

The passage 326 may include a seal member 328 for preventing fluid irrigation fluid from flowing out of the space outside of the inner aspiration catheter 304. The seal member 328 can provide less than 360 degrees of contact with the inner aspiration catheter 304. For example, the seal member 328 can have one or more lobes or prongs for interfacing with the inner aspiration catheter 304, for example two lobes or three lobes or four lobes. The separate contact points decrease the amount of friction between the inner aspiration catheter 304 and the seal member 328 and enable the use of a lower torque motor.

The inner aspiration catheter 304 is capable of being manually or automatically moved within the lumen of the support catheter. When automated, the catheter 150 may include a drive unit 330. The drive unit 330 may include a motor for driving the inner aspiration catheter 304 relative to the outer support catheter 302. The drive unit 330 may include or be operably connected to a controller configured to cause the motor to advance and retract the inner aspiration catheter 304. The drive unit 330 may include a battery source. The drive unit 330 may be a handheld component that is separately attachable to the inner aspiration catheter 304. For example, the same drive unit 330 may be used with disposable catheter assemblies.

FIG. 3B, on the other hand, illustrates the catheter 150 in an open position. As illustrated in FIG. 3B, the inner sheath 304 is extended toward the distal end 320 of the catheter 150 and protrudes out of the outer sheath 302. In addition, the valve 306 is open.

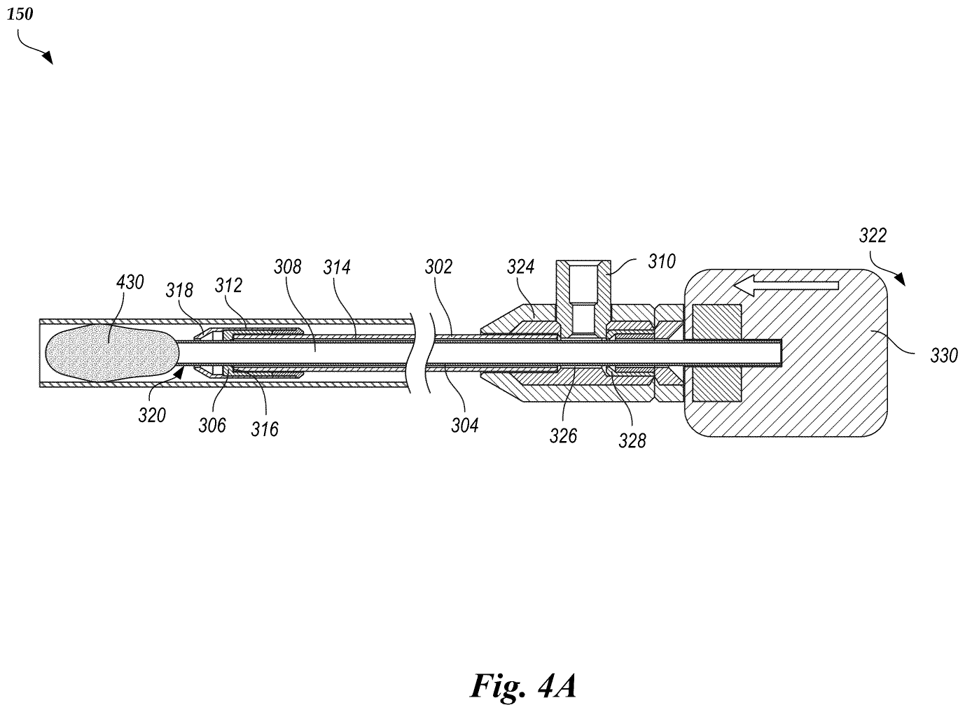

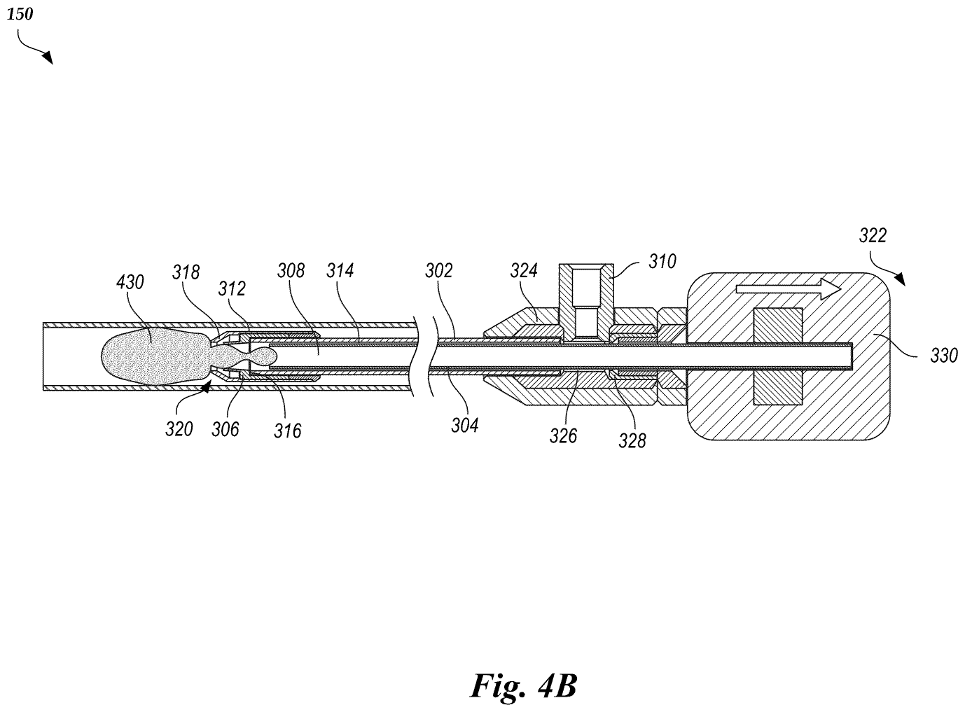

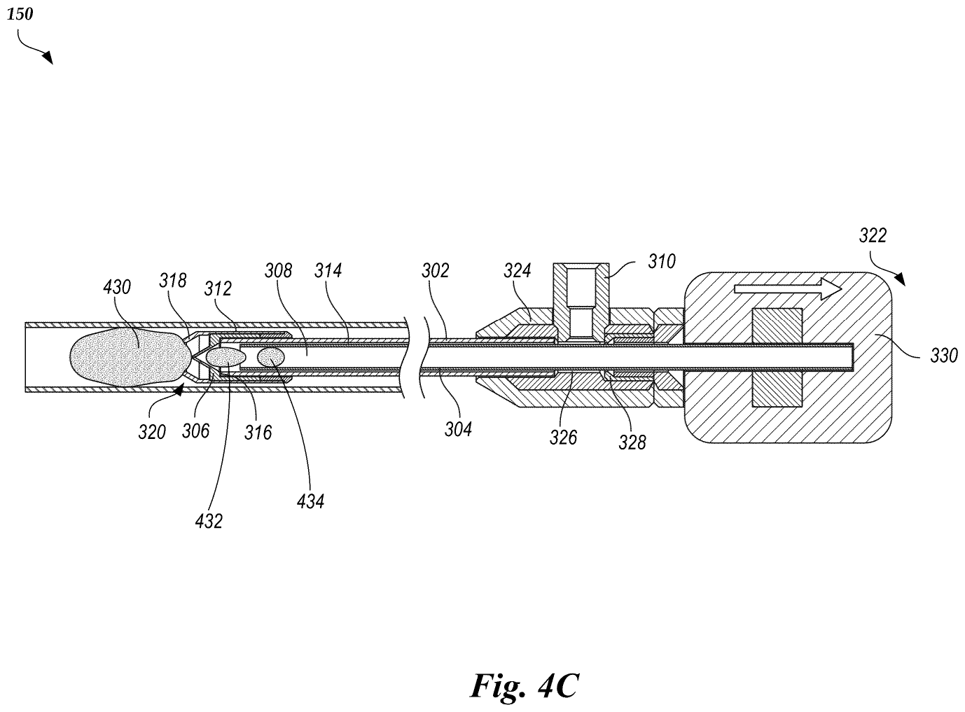

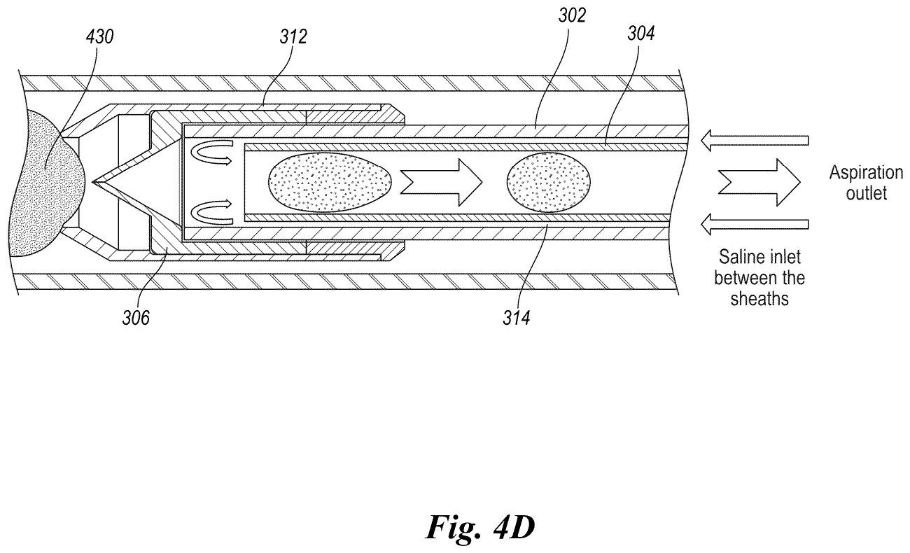

FIGS. 4A-4D illustrate an example catheter 150 being used in a procedure to aspirate a clot 430. As illustrated in FIG. 4A, the catheter 150 is in an open position, with the inner sheath 304 extended toward the clot 430, and is attempting to acquire the clot 430. As illustrated in FIG. 4B, the catheter 150 is in a retracted position, with the inner sheath 304 moving backward away from the distal end 320 of the catheter 150. Here, the clot 430 is extruded into the inner sheath 304 due to a vacuum force. The clot 430 may continue traveling in the inner sheath 304 toward the proximal end 322 of the catheter for storage in a collection canister. The amplitude by which the inner sheath 304 is extended to reach the open position and the frequency at which the inner sheath 304 oscillates between the open position and the retracted position may be determined by the trained artificial intelligence model.

As illustrated in FIG. 4C, the catheter 150 is in a retracted or flush position. For example, the inner sheath 304 may be positioned inside the outer sheath 302 such that the valve 306 is closed while clot pieces 432 and 434 (e.g., clot pieces segmented from the clot 430) are present in the inner sheath 304. The amplitude by which the inner sheath 304 is extended to reach the flush position and the frequency at which the inner sheath 304 oscillates between the flush position and the retracted position, if at all, may be determined by the trained artificial intelligence model. A vacuum force may pull the clot pieces 432 and 434 toward the proximal end 322 of the catheter 150, but the vacuum force alone may not be sufficient to pull the clot pieces 432 and 434 at a desired rate and/or to prevent clogging in the inner sheath 304. Saline injected in the inlet 310, however, may flow to the distal end 320 of the catheter and be pulled by the vacuum force. The saline may help prevent clogging in the inner sheath 304 because the force created by the saline pushing the clot pieces 432 and 434 and the vacuum force together may be sufficient to cause the clot pieces 432 and 434 to aspirate toward the proximal end 322 of the catheter 150 at least at a desired rate.

As illustrated in FIG. 4D, fluid flows between the outer surface of the inner aspiration catheter 304 and the inner surface of the outer support catheter 302. When the irrigation fluid reaches the valve 306, the irrigation fluid enters the distal end of the inner aspiration catheter 304 and pushes the macerated clot segments proximally through the inner aspiration catheter 304 as vacuum force aspires the clot segments. Adding water pressure can double the force of the vacuum to at least about 1 bar and/or less than or equal to about 2 bar. The added propellant speeds up aspiration and prevents clogging. The positive pressure may be applied using other methodologies, for example using a pump.

If the inner aspiration catheter 304 is clogged, the inner aspiration catheter 304 can be retracted proximal of the valve 306 without pulling the inner aspiration catheter 304 completely out of the body. This increases the suction of irrigation fluid. The irrigation flow will unclog the inner aspiration catheter 304. In some methods, irrigation flow may only be activated when non-continuous flow (clog) is detected or irrigation flow may be increased when non-continuous flow is detected. During this process, the inner aspiration catheter 304 may remain stationary or be advanced and retracted while still remaining behind the distal valve 306.

### Example Inner Sheath Actuation Routine

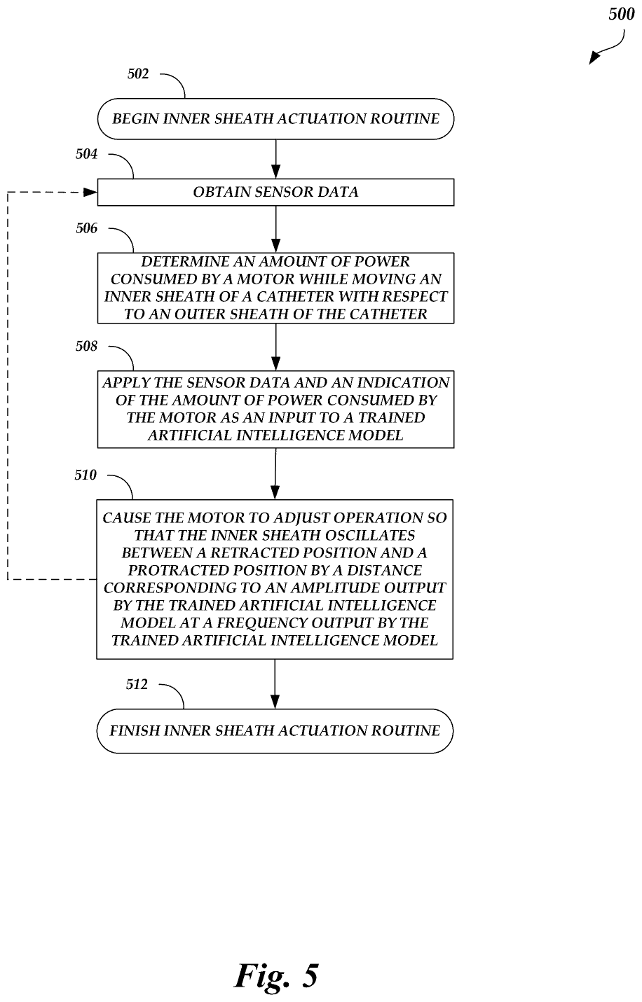

FIG. 5 is a flow diagram depicting an example, inner sheath actuation routine 500 illustratively implemented by a catheter control unit, according to one embodiment. As an example, the catheter control unit 120 of FIG. 1 can be configured to execute the inner sheath actuation routine 500. The inner sheath actuation routine 500 begins at block 502.

At block 504, sensor data is obtained. For example, the sensor data can include any of the sensor 155 measurements described herein. The sensor data may be data captured by one or more sensors 155 while the catheter 150 is being used in a procedure.

At block 506, an amount of power consumed by a motor while moving an inner sheath of a catheter with respect to an outer sheath of the catheter is determined. For example, the motor 140 may provide an indication of the amount of power being consumed. As another example, the motor 140 may provide a motor resistance and the amount of power being consumed can be derived from the motor resistance.

At block 508, the sensor data and the indication of the amount of power consumed by the motor are applied as inputs to a trained artificial intelligence model. As a result, the trained artificial intelligence model may output an amplitude and a frequency. Optionally, other data may be provided to the trained artificial intelligence model as well, including an indication of a type of valve present in the catheter 150 being used in the procedure and/or the type of motor 140 present in the catheter control unit 120 coupled to the catheter 150 being used in the procedure.

At block 510, the motor is caused to adjust operation so that the inner sheath oscillates between a retracted position and a protracted position by a distance corresponding to the amplitude output by the trained artificial intelligence model at the frequency output by the trained artificial intelligence model. As described herein, the amplitude could be 0 mm or the frequency could be 0 Hz, in which case the inner sheath does not oscillate until the trained artificial intelligence model is re-run and produces a different output, if applicable.

Blocks 504, 506, 508, and/or 510 may be repeated zero or more times during a single procedure. After the motor operation is caused to be adjusted, the inner sheath actuation routine 500 proceeds to block 512 and ends.

### Additional Embodiments

The network 110 may include any wired network, wireless network, or combination thereof. For example, the network 110 may be a personal area network, local area network, wide area network, over-the-air broadcast network (e.g., for radio or television), cable network, satellite network, cellular telephone network, or combination thereof. As a further example, the network 110 may be a publicly accessible network of linked networks, possibly operated by various distinct parties, such as the Internet. In some embodiments, the network 110 may be a private or semi-private network, such as a corporate or university intranet. The network 110 may include one or more wireless networks, such as a Global System for Mobile Communications (GSM) network, a Code Division Multiple Access (CDMA) network, a Long Term Evolution (LTE) network, or any other type of wireless network. The network 110 can use protocols and components for communicating via the Internet or any of the other aforementioned types of networks. For example, the protocols used by the network 110 may include Hypertext Transfer Protocol (HTTP), HTTP Secure (HTTPS), Message Queue Telemetry Transport (MQTT), Constrained Application Protocol (CoAP), and the like. Protocols and components for communicating via the Internet or any of the other aforementioned types of communication networks are well known to those skilled in the art and, thus, are not described in more detail herein.

### Terminology

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or logic circuitry that implements a state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements or steps. Thus, such conditional language is not generally intended to imply that features, elements or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A system comprising:
a catheter comprising an outer sheath and an inner sheath;
one or more sensors coupled to the catheter; and
a control unit coupled to the catheter and the one or more sensors, wherein the control unit comprises:
a motor configured to move the inner sheath with respect to the outer sheath; and
a processor configured with computer-executable instructions, wherein the computer-executable instructions, when executed by the processor, cause the processor to:
obtain sensor data from at least one of the one or more sensors;
determine an amount of power consumed by the motor while moving the inner sheath with respect to the outer sheath;
apply the sensor data and an indication of the amount of power consumed by the motor as an input to a trained artificial intelligence model, wherein application of the sensor data and the indication of the amount of power consumed by the motor as an input to the trained artificial intelligence model causes the trained artificial intelligence model to output an amplitude and a frequency; and
cause the motor to adjust operation so that the inner sheath oscillates between a retracted position and a protracted position by a distance corresponding to the amplitude at the frequency.

2. The system of claim 1, wherein the computer-executable instructions, when executed by the processor, further cause the processor to apply the sensor data, the indication of the amount of power consumed by the motor, and a type of the motor as an input to the trained artificial intelligence model.

3. The system of claim 1, wherein the catheter further comprises a valve at a distal end of the catheter configured to be inserted into a venous system.

4. The system of claim 3, wherein the computer-executable instructions, when executed by the processor, further cause the processor to apply the sensor data, the indication of the amount of power consumed by the motor, and a type of the valve included in the catheter as an input to the trained artificial intelligence model.

5. The system of claim 3, wherein the valve is closed when the inner sheath is in the protracted position.

6. The system of claim 3, wherein the valve is open when the inner sheath is in the protracted position.

7. The system of claim 3, wherein the trained artificial intelligence model is associated with at least one of a type of the valve or a type of the motor.

8. The system of claim 1, wherein the one or more sensors comprises at least one of a flow sensor, a contact sensor, a temperature sensor, a pressure sensor, or a camera.

9. The system of claim 1, wherein at least some of the one or more sensors are coupled to a distal end of the catheter configured to be inserted into a venous system.

10. The system of claim 1, wherein at least some of the one or more sensors are coupled to a proximal end of the catheter toward which one or more blood clot pieces are configured to be aspirated during operation of the catheter.

11. The system of claim 1, further comprising a catheter actuation training system configured with second computer-executable instructions, wherein the second computer-executable instructions, when executed, cause the catheter actuation training system to:
train an artificial intelligence model using training data to form the trained artificial intelligence model; and
cause the trained artificial intelligence model to be loaded onto a storage medium of the control unit.

12. A computer-implemented method for actuating an inner sheath of a catheter, the computer-implemented method comprising:
obtaining sensor data from at least one sensor coupled to the catheter;
determining an amount of power consumed by a motor configured to move the inner sheath with respect to an outer sheath of the catheter while moving the inner sheath with respect to the outer sheath;
applying the sensor data and an indication of the amount of power consumed by the motor as an input to a trained artificial intelligence model, wherein application of the sensor data and the indication of the amount of power consumed by the motor as an input to the trained artificial intelligence model causes the trained artificial intelligence model to output an amplitude and a frequency; and
causing the motor to adjust operation so that the inner sheath oscillates between a retracted position and a protracted position by a distance corresponding to the amplitude at the frequency.

13. The computer-implemented method of claim 12, wherein applying the sensor data and an indication of the amount of power consumed by the motor as an input to a trained artificial intelligence model further comprises applying the sensor data, the indication of the amount of power consumed by the motor, and a type of the motor as an input to the trained artificial intelligence model.

14. The computer-implemented method of claim 12, wherein the catheter further comprises a valve at a distal end of the catheter configured to be inserted into a venous system.

15. The computer-implemented method of claim 14, wherein applying the sensor data and an indication of the amount of power consumed by the motor as an input to a trained artificial intelligence model further comprises applying the sensor data, the indication of the amount of power consumed by the motor, and a type of the valve included in the catheter as an input to the trained artificial intelligence model.

16. The computer-implemented method of claim 14, wherein the valve is closed when the inner sheath is in the protracted position, and wherein the valve is open when the inner sheath is in the protracted position.

17. The computer-implemented method of claim 14, wherein the trained artificial intelligence model is associated with at least one of a type of the valve or a type of the motor.

18. A non-transitory, computer-readable medium comprising computer-executable instructions for actuating an inner sheath of a catheter, wherein the computer-executable instructions, when executed by a computer system, cause the computer system to:
obtain sensor data from at least one sensor coupled to the catheter;
determine an amount of power consumed by a motor configured to move the inner sheath with respect to an outer sheath of the catheter while moving the inner sheath with respect to the outer sheath;
apply the sensor data and an indication of the amount of power consumed by the motor as an input to a trained artificial intelligence model, wherein application of the sensor data and the indication of the amount of power consumed by the motor as an input to the trained artificial intelligence model causes the trained artificial intelligence model to output an amplitude and a frequency; and
cause the motor to adjust operation so that the inner sheath oscillates between a retracted position and a protracted position by a distance corresponding to the amplitude at the frequency.

19. The non-transitory, computer-readable medium of claim 18, wherein the computer-executable instructions, when executed, further cause the computer system to apply the sensor data, the indication of the amount of power consumed by the motor, and a type of the motor as an input to the trained artificial intelligence model.

20. The non-transitory, computer-readable medium of claim 18, wherein the catheter further comprises a valve at a distal end of the catheter configured to be inserted into a venous system, and wherein the computer-executable instructions, when executed, further cause the computer system to apply the sensor data, the indication of the amount of power consumed by the motor, and a type of the valve included in the catheter as an input to the trained artificial intelligence model.
